(19) United States
(12) Patent Application Publication
(10) Pub. No.: US 2023/0285080 A1
(43) Pub. Date: Sep. 14, 2023

(54) SYSTEMS AND METHODS FOR PROCEDURE PLANNING USING PREHABILITATION INPUT

(71) Applicant: INTELLIJOINT SURGICAL INC., KITCHENER (CA)

(72) Inventors: ANDRE NOVOMIR HLADIO, WATERLOO (CA); JOSEPH ARTHUR SCHIPPER, KITCHENER (CA)

(21) Appl. No.: 18/020,158

(22) PCT Filed: Aug. 6, 2021
(86) PCT No.: PCT/CA2021/051093

§ 371 Date: Feb. 7, 2023

Related U.S. Application Data
(60) Provisional application No. 63/062,894, filed on Aug. 7, 2020.

(51) Int. Cl. A61B 34/10; A61B 5/11

(52) U.S. Cl. CPC A61B 34/10; A61B 5/112; A61B 5/1128; A61B 2034/105; A61B 2505/09

(57) ABSTRACT

Currently, prehabilitation and pre-operative kinetic sensing is decoupled from surgical planning. Systems and methods are provided for procedure planning using data collected and provided by a prehabilitation (monitoring) system. Extrapolations (e.g. using a machine trained model or other approach) generate post-operative kinetic condition data predicting how the patient will move after surgery, based on the trends of their condition during prehabilitation, and the surgical intervention itself. A surgical plan optimized for the post-surgical kinetic condition of the patient may be generated and provided. The plan may be provided for display and/or to a surgical navigation or robotic system for the planned procedure. These and other aspects will be understood including a model, such as a neural network model, which is trained to provide the extrapolations.

### CROSS-REFERENCE

This application claims priority to U.S. Provisional Application No. 63/062,894, filed Aug. 7, 2020, the entire contents of which are incorporated herein by reference.

### FIELD

This application relates to computer systems and related methods for planning a surgical procedure, such as an anatomical reconstruction, to improve surgical outcome and more particularly to systems and methods for procedure planning using prehabilitation input.

### BACKGROUND

Prehabilitation in the context of surgery (for example, total joint arthroplasty (TJA)) is the concept of preparation to optimize a patient's physical condition for a surgical procedure and its recovery. For example, a patient's muscular strength, flexibility, range of motion, endurance, etc. may be optimized through a prehabilitation regimen prior to surgery, as these parameters may influence overall outcomes.

Sensors are increasingly used to measure patients' kinetic conditions before and after surgery. For example, wearable sensors such as the Apple Watch™ (Apple Inc, Cupertino, Calif.) are coupled with software for the purposes of pre and post-op patient monitoring and kinetic measurement (the MyMobility™ product marketed by Zimmer Biomet Inc, Warsaw, Ind.).

Existing surgical planning systems provide functionality to plan for desired anatomical reconstruction based on medical images of a patient. In total joint arthroplasty, the desired anatomical reconstruction is defined by implants (size, shape, type) and their positions relative to the patient's anatomy. Existing systems may use 2D or 3D medical images (for example, x-rays and CT scans respectively, among others). The general goal of surgical planning systems is to improve patient outcomes by planning for a desirable anatomical reconstruction. Currently, surgical planning is known to improve patient outcomes to a degree; however, there remain a proportion of patients with poor outcomes, despite the use of existing surgical planning systems.

There exists a need to improve surgical planning systems.

### SUMMARY

Systems and methods are provided for procedure planning using data collected and provided by a prehabilitation (monitoring) system. Extrapolations (e.g. using a machine trained model or other approach) generate post-operative kinetic condition data predicting how the patient will move after surgery, based on the trends of their condition during prehabilitation, and the surgical intervention itself. A surgical plan optimized for the post-surgical kinetic condition of the patient may be generated and provided. The plan may be provided for display and/or to a surgical navigation or robotic system for the planned procedure. These and other aspects will be understood including a model, such as a neural network model, which is trained to provide the extrapolations.

In an embodiment, there is provided a computer implemented method for pre-operative planning a TJA for a patient. The method comprises: receiving from a prehabilitation system first and second condition data, wherein the first condition data and second condition data comprising kinetic information about how the patient moves from two different time points; wherein the kinetic information is derived from spatial measurements of the patient's movement; and, wherein the two different time points are separated by sufficient time to allow for detectable physiological changes relating to the patient's kinetic condition; extrapolating a post-surgical kinetic condition of the patient based on the first and second condition data and the planned surgical intervention; calculating a surgical plan optimized for the post-surgical kinetic condition of the patient; and providing the plan for display.

Computer device and computer program aspects are also provided.

### BRIEF DESCRIPTION OF THE DRAWINGS

FIG. 1 is a block diagram of a network system comprising a plurality of respective computing devices, in accordance with an embodiment.

FIG. 2 is an example of a graphical user interface (GUI) for surgical planning, in accordance with an embodiment, which GUI is presented by a surgical planning computing device of FIG. 1.

FIG. 3 is an illustration of data coregistration components and operations in accordance with an embodiment to coregister respective medical image data (e.g. a medical image) and sensor data obtained for a patient where each of the medical image and sensor data have respective coordinate systems.

FIGS. 4A and 4B are example GUIs for surgery scheduling, in accordance with an embodiment.

FIGS. 5, 6 and 7 are flowcharts of respective operations of a computing device illustrating respective computer-implement methods in accordance with embodiments.

### DETAILED DESCRIPTION

### High Level Overview

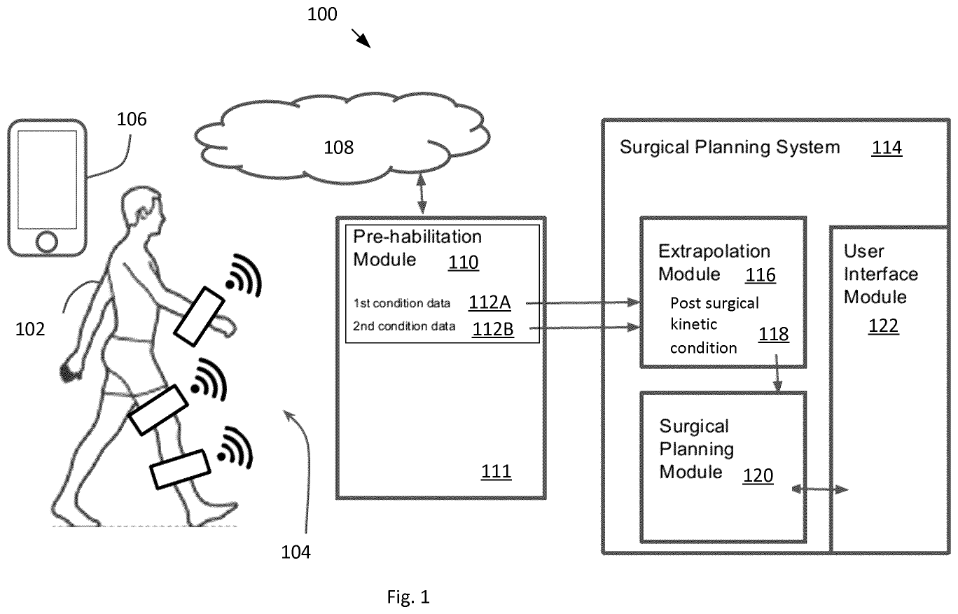

FIG. 1 is a block diagram and illustration of a sensor-based network system 100 (e.g. abbreviated to system 100) for procedure planning such as for prehabilitation, in accordance with an embodiment. In an embodiment, a patient 102 is a candidate for a musculoskeletal surgery, in which the kinetic condition of the patient 102 is to be altered. For example, total joint arthroplasty (TJA) surgeries, such as hip and knee replacements, seek to improve the overall kinetic condition of the patient through increased range of motion, pain free/reduced movement, and/or restoration of normal biomechanics. In another example, the surgery may be a spinal fusion, in which the overall kinetic condition of the patient is improved (e.g. pain free/reduced movement), through limiting the range of motion between diseased vertebra.

In an embodiment, system 100 is used for prehabilitation of the patient 102, for example, where the patient 102 uses wearable sensors 104 coupled to the patient's body (e.g. their limb segments). The system 100 for prehabilitation may use any type of sensor, and in combination with sensor data processing functionality, may measure a patient's pre-operative condition, including their kinetic condition. The system 100 for prehabilitation may include sensors 104, and a plurality of computing devices comprising a mobile device 106, a cloud server 108, a prehabilitation module 110 (e.g. implemented on a computer server 111 or other computing device) and a surgical planning system 114.

The wearable sensors 104 may communicate with the mobile device 106, for example, using wireless communication such as Bluetooth™ or WiFi. The mobile device 106 may communicate with the cloud server 108 using wireless communication such as a cellular network connection, or combination of networks (wired and/or wireless).

In an embodiment, the mobile device 106 also provides sensor measurements of the patient's pre-operative condition, including their kinetic condition, for example, using cameras, inertial sensors or global positioning system (GPS) sensors available on commonly available existing mobile devices. The camera may be a component of a motion capture system (e.g. making use of fiducials attached to patient's body tracked by the camera). Pressure sensors, including pressure mats, may be used to collect and provide kinetic condition data.

In an embodiment, the mobile device 106 also provides a user interface for the patient, to aid in such functions as: pairing the mobile device 106 and the sensors 104; providing a prehabilitation system client application to the patient; tracking and displaying prehabilitation metrics; and data entry of pre-operative condition data (e.g. forms, checklists, surveys, journals etc.).

In an embodiment, sensor data, and other condition data, collected by wearable sensors 104 and/or the mobile device 106, is provided to a prehabilitation module 110 (for example, through a cloud server 108. The prehabilitation module 110 may be implemented on a single computer device, or may be distributed amongst multiple devices. For example, the prehabilitation module 110 functionality may be distributed amongst a dedicated server, the mobile device 106, the cloud server 108, and the surgical planning system 114. Other configurations may be used.

In an embodiment, sensor data and other condition data is processed to ensure data quality and standardization. For example, the prehabilitation module 110 may perform data processing operations such as signal conditioning (e.g. low-pass filtering), and outlier rejection (e.g. implementing random sample consensus (RANSAC) algorithms). Further data processing may include converting the sensor and condition data into a standardized data format such as comma-separated values (CSV) or JavaScript Object Notation (JSON). In an embodiment, sensor data and other condition data includes time information (i.e. each datapoint may be associated with when it was collected, such as by using a time stamp).

In an embodiment, sensor or other condition data from the same, or similar activities is received by the prehabilitation module 110 over time. For example, the sensor data may be generated by wearable sensors 104, and measuring the range of motion of the patient's knee, and provided to the prehabilitation module 110. Several weeks later (by way of example), the same range of motion activity may be performed, and sensor data may again be generated by the wearable sensor 104, and provided to the prehabilitation module 110. Similarly, a pain survey may be completed via the mobile device 106 at two different points in time, separated by enough time for physiological changes relating to a prehabilitation regimen to occur, and provided to the prehabilitation module 110.

In an embodiment, eventually sufficient data is generated for the purposes of extrapolation. To enable extrapolation, the data may be required to be based on the same or similar patient activities from two different time points, separated by at least enough time to allow for any physiological changes from the prehabilitation regimen to take place. For example, it may take at least two weeks to develop changes in strength or range of motion. Further qualities of the data for extrapolation may include: data confidence such as confidence calculated from multiple repeated measurements, data reliability such as data recorded by a medical professional vs sensor generated data vs self-reported values, limiting interacting factors such as activities performed after injury, or activities performed at different temperatures or sickness levels. At minimum, the data for extrapolation may include a first condition data 112A and a second condition data 112B, where both data are from the same or similar patient activities from two different and sufficiently separated points in time. Practically, pre-operative condition data may be from a multitude of time periods, under a widely varying range of activities.

In an embodiment, the preoperative kinetic and other condition data for extrapolation (e.g. first condition data 112A and second condition data 112B) is received by an extrapolation module 116 of surgical planning system 114. The surgical planning system 114 may be implemented on a single computer device, or may be distributed amongst multiple devices, including on devices common to the prehabilitation module 110. The extrapolation module 116 performs computations on the first condition data 112A and second condition data 112B and outputs a prediction of the patient's post-surgical kinetic condition 118. The post-surgical kinetic condition 118 is used as an input to a surgical planning module 120. A user interface module 122 is provided to enable a user (for example, a surgeon) to perform surgical planning, by providing information, visualizations and controls for user interaction. Post-surgical kinetic condition data 118 includes information that is spatial in nature, predicting how the patient will move after surgery. The surgical planning module 120 may also receive 2D or 3D medical images of the patient (i.e. of the anatomical structures to be altered or reconstructed during surgery). The surgical planning module 120 may provide spatial targets to the surgeon, via the user interface 122, based on the desired reconstruction. The spatial targets may be in the form of graphical overlays on medical images, for example, of implants, numerical information, graphical information, manipulations of medical images (for example, repositioning of selected image features, such as realigning pixels or voxels of a crooked bone to be straight), annotations, etc.

### ——Kinetic/Condition Data——

Kinetic data generally refers to data associated with how a patient moves in a spatial sense (that is, how a patient moves in 3D space). Examples of kinetic data include: ranges of motion of joints, gait, posture, stance, and the kinematics of activities of daily living (ADL), such as getting in or out of a vehicle, mounting stairs, using the toilet, picking an item up off the floor, etc. The spatial dimensions or units of kinetic data include angular and translational positions (i.e. and orientations), velocities and accelerations, as well as measures of quantity such as duration (e.g. duration of a particular movement or activity), frequency (e.g. repetition of a particular movement) and count (e.g. how many repetitions, such as number of steps taken in a day). Kinetic data may have associated contexts. For example: a passive versus an active range of motion; pain free movement versus movement with pain; level of relaxation; etc.

Condition data generally refers to a broader set of data associated with or influencing how a patient moves, and may include kinetic data. Condition data may include kinesiological data for the patient, including physiological, psychological, biomechanical, anatomical, and neurological data. Condition data may also include social, emotional, demographic (e.g. race, gender, BMI (body mass index), age, height, weight, etc.) data. Condition data may also include information about a patient's modifiable risk factors (such as smoking, alcohol or drug use), comorbidities (such as diabetes, neurological disorders, previous trauma, mental health issues such as depression and anxiety), pain scores, prehabilitation compliance metrics (e.g. an indicator of how well the patient is complying with the prescribed prehabilitation regimen), and patient reported outcome measures such as the Knee injury and Osteoarthritis Outcome Score (KOOS), Hip disability and Osteoarthritis Outcome Score (HOOS), the Western Ontario and McMaster Universities Osteoarthritis Index (WOMAC), the Harris Hip Score (HHS) or other quality of life scores. Condition data may also include psychometric data for the patient, based on psychometric tests, such as the SF-36 Health Status Questionnaire. Psychometric data may represent a patient's mental and psychological factors that influence their current or future movement. For example, a psychometric test may reveal that a patient's motivation level is low, or that they have an intense fear of pain.

Condition data may also include information about the patient's planned future medical procedures (e.g. surgical interventions). For example, the patient may be planning for an additional surgery after the one for which they are prehabilitating (e.g. they may be considering a spinal fusion after their total hip arthroplasty). Such data may include a listing of future surgical procedures, and a binary flag to indicate whether they are considering that procedure after the one for which they are currently preparing.

Condition data may also include information about a patient's strength and/or endurance. For example, muscle strength or endurance may be quantified as the number of repetitions and resistance level of an exercise using an exercise machine or a weight. The condition data may be measured (for example, using a force measuring device) or manually entered into a data capture interface of the prehabilitation module 110. Condition data may also be data about a patient's cardiovascular health, such as a patient's cholesterol profile, hemoglobin A1C level, stress test results, etc.

Condition data may also include a patient's dietary information, such as the results of a food journal, dietary restrictions, types and doses of vitamins and supplements, etc. Condition data may also include information about a patient's medications (such as what medications they are currently taking for what purpose).

Condition data may be received by the prehabilitation module from any source, including from sensors (such as wearable sensor 104 or sensors within the patient's mobile device 106), data entry (for example, forms, surveys, tests, data fields, notes, provided by a prehabilitation client application on the patient's mobile device 106), and from the patient's medical records (for example, by connecting to the patient's electronic medical records).

In accordance with an embodiment, condition data preferably has associated time-based information (for example, a psychometric test may be administered on a particular date, that date being associated with the results of the test and forming part of the condition data). Similarly, a patient's weight may form part of the condition data, along with the date on which that weight was recorded. Condition data preferably is based on activities (such as weighing or taking a test) that may be repeated in the future, for the purposes of trending and extrapolation.

### ——Sensor Types——

Different types of sensors may be employed by systems for prehabilitation. Sensors may measure data relating to a patient's kinetic condition, or relating to condition data more broadly. To measure a patient's kinetic condition, sensors that measure a patient's movement are preferred. Such sensors may include inertial sensors, such as inertial measurement units, accelerometers, gyroscopes, magnetometers. Such sensors may include optical sensors, such as cameras, including infrared cameras, visible spectrum cameras, and depth cameras (such as time of flight cameras). Cameras may capture a position of fiducials attached to the patient. Such sensors may include optical sensors and structured light projectors. Such sensors may include GPS sensors. Such sensors may include pressure sensors, including pressure mats. Such sensors may include electro-magnetic sensors. Such sensors may include resistance sensors such as goniometers.

A system for prehabilitation may employ any combination of the aforementioned types of sensors, including multiples of the same type of sensor. The sensors may be used to measure a patient's kinetic condition by being coupled to anatomical structures of the patient during movement (e.g. inertial sensors may be strapped to a patient's limb segments while they are performing a prescribed motion). The sensors may measure the patient's kinetic condition without patient contact, for example, optically. A camera system may be configured to measure the pose of a patient (conducting a prescribed motion) within its field of view. The pose of the patient may include the poses of each individual relevant body segment of the patient. The above examples demonstrate that there are many ways in which sensors may be used to measure the kinetic condition of a patient, and this description considers all possible ways of measuring a patient's kinetic condition without limitation to specific technologies or implementations, whether now existing or in the future.

In addition to sensing a patient's kinetic condition, sensors may be used to measure other aspects of the patient's condition. For example, heart rate monitors, electromyography (EMG) sensors, blood oxygen sensors, cameras, force sensors, sleep quality sensors, temperature sensors, any of the aforementioned types of sensors for measuring kinetic conditions, or any other type of sensor may be used, where applicable, to measure any aspect of the patient's condition (including the first condition data 112A and the second condition data 112B).

The term “sensor” does not strictly mean a single sensor, but may mean a collection of coupled sensors. Furthermore, the term sensor (whether used for an individual sensing device, or multiple coupled sensing devices) may include analog and/or digital devices for signal processing, data processing and transmission. For example, a Bluetooth (Bluetooth SIG, Inc., Kirkland, Wash., U.S.A.) capable inertial measurement unit may be considered a sensor, though it is comprised of a 3 orthogonal accelerometers and 3 orthogonal gyroscopes; it may include on-board signal processing to co-register the accelerometers and gyroscopes, and on-board processing and radio devices to transmit the sensor data wirelessly using Bluetooth-based wireless communication.

### ——Extrapolation Module——

In accordance with an embodiment, the extrapolation module 116 may be implemented as computer instructions to execute on a computing device (e.g. a device of system 114). The extrapolation module 116 receives as input first condition data 112A and second condition data 112B, and outputs the post-surgical kinetic condition 118, which includes data describing how the patient is predicted to move after surgery. In an embodiment, parameters of the surgery are provided to or defined within the extrapolation model to define the surgical intervention for use to determine the post-surgical kinetic condition. The extrapolation module 116 may implement a computer model that describes human movement under a variety of conditions, including under the conditions musculoskeletal disease state (e.g. osteoarthritis of the hip).

The computer model may be implemented as a physics-based model based on the kinematics and dynamics of human body movement. For example, the computer model may be implemented as a parameterized dynamic model of a human body (parameters such as weight, length, width, height, strength, moments of inertia, center of mass, joint angles, etc. may be associated with individual or collective body segments). The extrapolation module 116 may determine pre-operative patient specific computer models based on patient specific data and first condition data 112A and second condition data 112B, specific to the patient 102.

These pre-operative patient specific computer models include received or calculated parameters for the respective models (where parameters are calculated, optimization routines may be used to minimize cost functions such as cost functions to minimize the required energy of one or more specified motions or cost functions to maximize joint stability for one or more motions or positions, while respecting physical constraints).

A first set of parameters (associated with the first condition data 112A) and a second set of parameters (associated with the second condition data 112B) may be determined by the extrapolation module 116. Physics-based and/or statistical methods, in combination with further optimization routines to ensure accurate and feasible movements, may be implemented by the extrapolation module 116 to calculate predicted post-surgical parameters.

To calculate post-surgical parameters, parameters associated with the surgical intervention may be changed (reflecting the effect of the surgical procedure). Such surgical intervention parameters may be stored in memory, such as in a list or database. Surgical intervention parameters are data that indicate any type of surgical intervention that may be considered for a given disease (for example, if the disease state is right hip osteoarthritis, surgical intervention parameters may include: right total hip replacement, right hip resurfacing, right peri-acetabular osteotomy, right hip arthroscopy, right partial hip replacement, etc.). The surgical approach (e.g. direct anterior total hip arthroplasty) may be specified as a type of surgical intervention parameter. The change in surgical intervention parameters reflect parameters that are subject to change dramatically due to the surgical intervention, but are unlikely to change significantly during or due to the prehabilitation regimen. For example, a stiff hip is likely to remain stiff during prehabilitation, but will become (more to) fully mobile after surgery. Because of the changes imparted to the model by the surgical intervention parameter, it may be preferable to perform further optimization routines that include both the changes due to the surgical intervention parameter and the changes based on extrapolating the other parameters.

For example, where the extrapolation module implements a computer model modelling a patient's movement, the surgical intervention parameters may be used to modify the computer model to reflect changes resulting from the particular surgical intervention (e.g. muscle weakness due to the surgical approach of a total joint arthroplasty procedure may be reflected in the computer model by changing coefficients associated with particular muscle strengths). Surgical intervention parameters may have similarities to first and second condition data, in that intervention parameters are an input into the extrapolation computation. Intervention parameters represent changes that are due to the modification of tissues (in an example) in surgery (whereas the first and second condition data relate (partially) to modification of tissues through normal biological processes—e.g. strengthening muscles). As such, the extrapolation takes as inputs the “surgical” and “biological” modifications to predict the post-surgical condition. Surgical intervention parameters may relate to modification of bone. Surgical intervention parameters may be associated with changes to range of motion, pain, strength and other characteristics.

In an embodiment, extrapolation may account for predicted additional progress achieved through biological processes before surgery, such as additional strengthening, weight loss, mobility gains, etc. Additional progress may be achieved by continued physical therapy, diet, exercise, etc. In an embodiment, a model may extrapolate initial results (e.g. a trend) such as presented by the first and second condition data.

In an embodiment, the extrapolation results of a post-surgical condition may be presented to a patient to encourage continued or improved adherence to pre-surgery therapies such as but not limited to physiotherapy or weight loss therapy. In an embodiment, the extrapolated post-surgical condition represents a marked improvement in kinetic condition, etc. Providing these results to the patient may encourage continued adherence. In an embodiment, the extrapolated post-surgical condition represent negligible or very small improvement, at least suggesting that unless the patient's pre-surgical condition is improved, the post-surgical condition may be insufficient to warrant surgery (e.g. in the short term). In an embodiment, extrapolation results of a post-surgical condition are useful to determine a date (e.g. a time range) for surgery, such as described elsewhere herein.

The extrapolation module 116 may implement a model based on machine learning. The model may receive the first condition data 112A, second condition data 112B, and surgical intervention parameters and calculate an expected post-surgical condition. The model may be based on a neural network consisting of an input layer, one or more hidden layers, and an output layer with weighted connections between the nodes in each layer. The model may be a recurrent neural network which receives a plurality of (more than two) instances of condition data and processes the instances (e.g. in a sequential nature) to calculate an expected post-surgical condition. The model may be designed according to other neural network architectures not described here. Other machine learning frameworks may be considered such as linear, non-linear regression or statistical techniques.

The post-operative kinetic condition 118 preferably includes data predicting how the patient will move after surgery, based on the trends of their condition during prehabilitation, and the surgical intervention itself. For example, the post-surgical kinetic data 118 may include: joint range of motion (including the range of motion of a reconstructed joint), standing patient posture, sitting patient posture, parameters of a patient specific dynamic model or activity level (e.g. duration, intensity of post-operative activities).

In any of the described implementations, the extrapolation module 116 may include the surgery date in any extrapolation computations. The surgery date may be data received by the extrapolation module 116, modifiable via the user interface module 122. The extrapolation module can use the surgery date as a constraint in any optimizations, or alternatively, the surgery date may be considered a variable to be calculated (e.g. optimal date of surgery for best predicted outcome). The surgery date per se may not be particularly relevant, but the delta such as from one or both of the first and second condition data (which include dates/timestamps) to the surgery date is a measure of time. In an embodiment, the module 116 extrapolates over time. Time need not be finely granular (e.g. to the minute). It may be sufficient to know the surgery date by its month, quarter or year (not the exact date). It could also be a range in time (for example, surgery is between April and October 2022), or even an unbounded range (surgery to occur after April 2022).

The types of models within the extrapolation module 116 described herein are provide by way of non-limiting examples. Aspects of the models as described may be combined. Other types of models are contemplated as well.

### ——Training System for AI-based Extrapolation Module——

A machine learning model implementing a conventional neural network may be trained with a training system (e.g. a computing device). The training system is configured to receive multiple instances of training data and return a trained model such as a classifier. In accordance with an embodiment, each instance of training data comprises a first condition data, a second condition data, and a measured post-operative kinetic condition data for a person. Training data may additionally comprise recorded surgical intervention parameters such as parameters described previously corresponding to implemented surgical interventions. The training system may implement machine learning techniques such as neural networks, linear or non-linear regression, or statistical techniques corresponding to the trained model design to identify critical parameters and parameter interactions of the first and second condition data and their relationships to the recorded post-operative condition data. Additional data instances may define validation or test data with which to test the model developed using data instances that define the training data.

The results of training may identify a level of importance for kinematic parameters such as indicating that some parameters are critical to extrapolate the post-operative kinetic condition 118 and that other parameters do not influence the post-operative kinetic condition 118. The training data may comprise a plurality of instances of condition data when the trained model is a recurrent neural network.

### ——Planning Module Detail——

In an embodiment, surgical planning module 120 receives the post-surgical kinetic condition 118 of the patient, and provides surgical planning functionality to a surgeon via a user interface (e.g. via the user interface module 122). Surgical planning functionality may focus on spatial planning, and include parameters such as: anatomical angles, anatomical distances, implant sizes, implant make, implant model, implant style, implant position and/or angle with respect to anatomical structures, etc. For example, surgical planning may include templating functionality, such the functionality provided by systems such as the TraumaCAD™ system (Brainlab AG, Munich, DE). Surgical planning may include dynamic or kinematic analyses, such as those offered in the Corin OPS™ product (Corin Group, Cirencester Gloucestershire, UK). The surgical planning module preferably receives pre-operative or intra-operative medical images, such as x-rays, magnetic resonance imaging (MRI) scans, computed tomography (CT) scans, ultrasound, intra-operative fluoroscopy, or any other modality useful for spatial surgical planning (i.e. planning spatial aspects of the surgical intervention relative to anatomical structures). The surgical planning module 120 may receive medical images using the Digital Imaging and Communications in Medicine (DICOM) standard, or any other standard. The surgical planning module 120 may be configured to generate a surgical target or range, representing the desired spatial, biomechanical or reconstructive changes due to the surgical procedure.

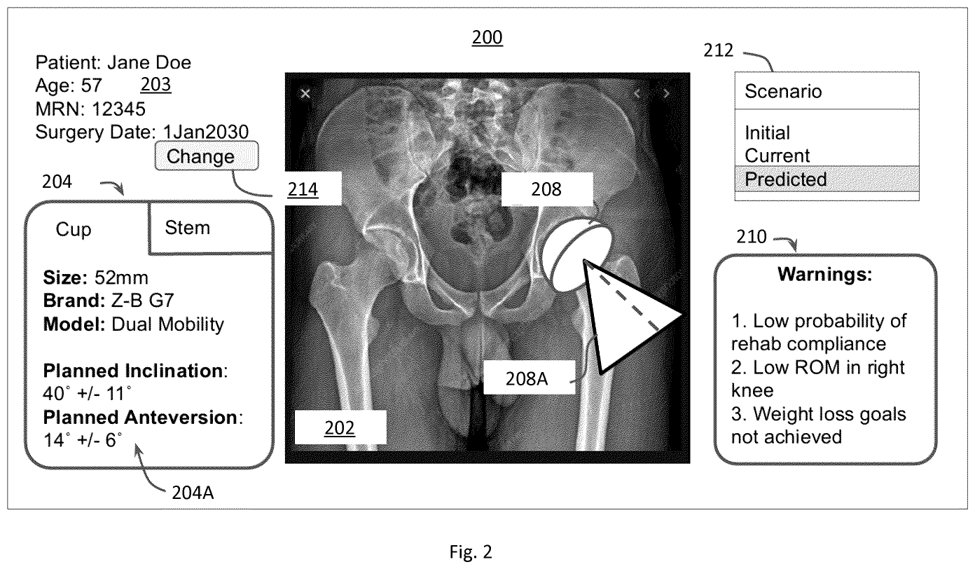

FIG. 2 depicts a user interface (e.g. a GUI) 200 in accordance with an embodiment. The user interface 200 is configured to display a medical image 202 of a patient (by way of example, 102) for whom the surgery is planned. Additional patient details 203, such as name, medical record number (MRN) and surgery date are shown. Planning parameters 204, such as the implant make, model and size are shown. The planning parameters 204 may include surgical target information 204A, such as a target inclination and anteversion for an acetabular prosthesis for a hip replacement surgery. The planning parameters 204 may be for multiple implants, such as a cup and a stem for use in a total hip arthroplasty. The surgical target information 204A may be presented numerically, for example, as a range of acceptable values for a particular spatial parameter (for example, the acceptable range of acetabular inclination relative to a planning coordinate frame may be between 29° and 51°, or equivalently, 40°+/−) 11°. Alternatively or in addition, the surgical target information 204A may be presented graphically, as shown in target graphic 208B, in which the shape of the target graphic 208B illustrates angular boundaries of the surgical target 204A relative to the current position of an implant overlay 208 (which includes a dotted line indicating the implant axis, the dotted line lying within the target graphic 208B indicating that the current implant position as indicated by the overlay is within the surgical target zone).

In accordance with an embodiment, the surgical planning system 114 facilitates a user to interact with the user interface by providing various controls to conduct planning (such as handles on graphical overlays, such as implant overlays, buttons, data capture fields, menus, etc.

In accordance with an embodiment, the surgical planning module 120 generates planning parameters 204 and surgical target information 204A by implementing optimization operations, for example, using linear, non-linear, or integer optimization techniques. Clinically and physically relevant cost functions and constraints may be implemented in optimization operations. For example, an optimization cost function may include minimizing a Euclidean norm associated with boney or implant impingement for a total joint replacement. A cost function may include minimizing spatial deviation from a kinematic trajectory representing healthy joint kinematics for total joint replacement (i.e. to optimize the surgical plan for natural movement post-operatively). A cost function may include minimizing edge loading of implants, to prevent premature wear. For a total joint arthroplasty procedure, optimization constraints may include: available makes and models of implants; physical constraints; elements of the post-surgical kinetic condition 118 etc. Furthermore, elements of the post-surgical kinetic condition 118 may be used as parameters for optimization operations. Any constraint or cost function that is clinically and physically relevant to the surgical procedure and the spatial goals of surgical planning may be used.

In addition to, or instead of generating surgical target information 204A or target graphics 208B, in accordance with an embodiment, the surgical planning module generates and provides for display warning information 210. Warning information 210 is information that is clinically relevant to the surgical planning process, which may not affect surgical target information 204A, target graphics 208B or surgical planning information 204. The warning information 210 may comprise elements of, or be derived from the post-surgical kinetic condition 118. For example, the warning information could include information about the health of non-operative joints, information about how the patient has performed during prehabilitation, or any other information. For example, the post-surgical kinetic condition for a right total hip arthroplasty patient may include warning information 210 (as shown in FIG. 2) stating that 1. The patient has a low probability of rehabilitation compliance, 2. The patient has low range of motion in their left knee, and 3. The weight loss goals of prehabilitation were not achieved. The warning information may be clinically relevant to a surgeon's planning process, in which the details of the surgical intervention, including implant size, make, model, position, etc. are planned. The warning information 210 may result from optimization operations performed by the surgical planning module 110, as previously described.

In accordance with an embodiment, the surgical planning module 110 receives first condition data 112A and second condition data 112B, and may further be configured to provide multiple surgical plans (i.e. surgical target information 204A and/or planning parameters 204) the respective patient conditions associated with the first condition data 112A, second condition data 112B and post-surgical kinetic condition 118. A user interface, in accordance with an embodiment, facilitates a user to select between the different patient conditions (e.g. using a dropdown menu 212 or other interface control type), or may present multiple plans associated with respective patient conditions simultaneously. The advantage of allowing a user (such as a surgeon) to view surgical plans associated with different patient conditions is to provide insight, for example, into the utility of the surgical planning system 114 or into how effective the prehabilitation regime has been for the patient.

In accordance with an embodiment, the surgical planning module 110 receives the post-operative kinetic condition 118 from the extrapolation module 116 based on a particular surgery date. That date may be provided for display via the user interface module 122. The user interface may include a date input function (such as indicated by a “Change” button 214 (a UI control)), in which a user can enter a new surgical date. Upon entering a new surgical date, in accordance with an embodiment, the surgical planning module 110 transmits the new date to the extrapolation module 116, which in turn updates the post-surgical kinetic condition 118 and the surgical plan for display (e.g. the surgical target information 204A, the planning parameters 204, and the warning information 210).

Examples of implant types that are considered planning parameters include: dual mobility implants (for total hip arthroplasty) and medial pivot implants (for total knee arthroplasty). Planning parameters 204 may also include surgical approach information (such as the direct anterior approach for total hip arthroplasty), surgical technique information (such as kinematic alignment in total knee arthroplasty).

In accordance with an embodiment, the surgical planning module 120 feeds back data relating to the surgical plan (e.g. planned implant positions) to the extrapolation module 116 for recalculation of the post-surgical kinetic condition 118. Such a feedback loop may proceed iteratively, responsive to a user's changing of plan information via the user interface of the surgical planning system 114.

### ——Coregistration Detail——

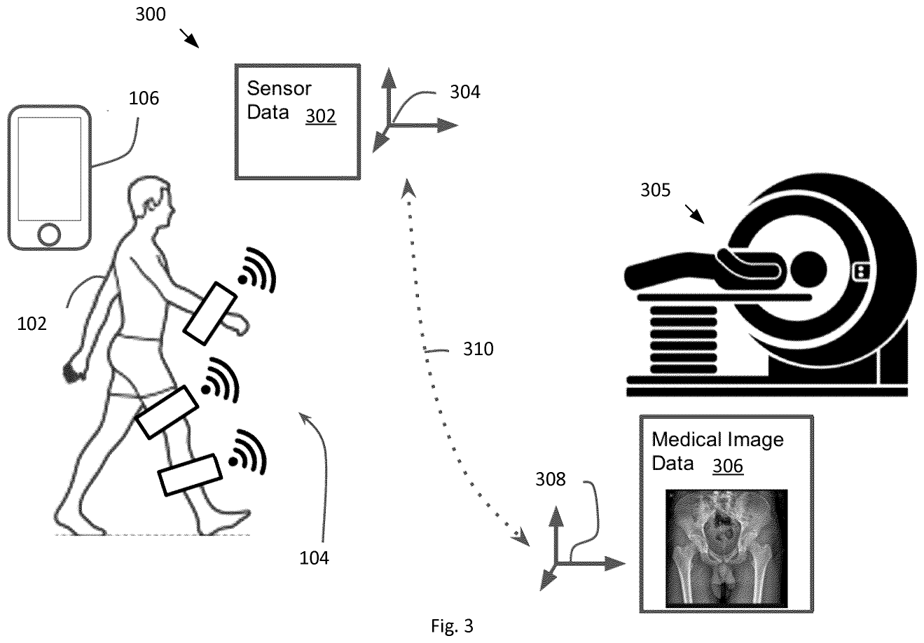

FIG. 3 is an illustration of data coregistration components and operations 300 in accordance with an embodiment. With reference to FIG. 3, sensor data 302 (e.g. from the wearable sensors 104 of the patient 102 undergoing prehabilitation) may have an association with a sensor data coordinate system 304. That is, the spatial data that is collected by the wearable sensors 104 (or any sensors associated with the prehabilitation regimen) may be expressed within a defined 3D coordinate system (or coordinate frame). Prior to surgery, the patient 102 may undergo medical imaging scans (such as x-ray, CT, MRI, etc.), via an imaging device 305, for diagnostic and pre-operative planning purposes, resulting in medical image data 306. The medical image data 306 may have an association with a medical image coordinate system 308. There exists a registration relationship 310 between the sensor data coordinate system 304 and the medical image coordinate system 308. The registration relationship may be a rigid body transformation from one coordinate system to the other, and may be expressed mathematically as an affine transformation matrix.

Coordinate systems (e.g. the sensor data coordinate system 304 and medical image coordinate system 308) may be orthogonal Cartesian coordinate systems. Each coordinate system may be defined by the location of its origin and the direction of the basis vectors. Both coordinate systems may represent their respective data (from spatial prehabilitation sensor data and medical image data) in coordinate systems defined by the patient. For example, the patient 102 may have biomechanical or anatomical axes or locations used to define both the sensor data coordinate system 304 and the medical image coordinate system 308. In another example, the two coordinate systems may be defined differently (i.e. with respect to different anatomical locations or axes), but the different coordinate system definitions may be relatable through rigid body transformations that are known or determinable.

The registration relationship 310 enables the sensor data 302 and the medical image data 306 to be expressed in a common coordinate frame relative to the patient 102. This is advantageous, since both the medical image data 306 and the sensor data 302 (via the post-surgical kinetic condition 118) may be used by the surgical planning module 120. Both coordinate systems may be relatable to a patient coordinate system, such as the standing coronal plane, supine coronal plane, and anterior pelvic plane. The planning module 120 may use the sensor data 302 and medical image data 306 relative to a common coordinate system to provide surgical planning functionality (e.g. offering a user interface in which the medical image and sensor data, or data derived therefrom, may be visualized and/or manipulated in the same view or canvas), or perform surgical planning steps (e.g. an optimal target implant position may be calculated relative to the common coordinate system (or frame), during which the surgical planning module 120 may perform spatial optimizations using the medical image data 306 and sensor data 302 within the same coordinate system).

Converting the sensor data 302 to a common coordinate system with the medical image data 306 (or vice versa) may be done using mathematical operations using a computer system (in particular, by applying rigid body transformation operations using the registration relationship 310). In reference to FIG. 1, such operations may be performed by the surgical planning system 114, including, for example, by the surgical planning module 120. The operations may be performed by first determining the medical image coordinate system 308 and the sensor data coordinate system 304, which may be done in any of the following manners: the respective data may be in an inherent or assumed coordinate system (e.g. the supine coronal coordinate system for a CT scan), the respective data may include further data defining the coordinate system (e.g. fiducials present within an x-ray scan, wherein the fiducials define the coordinate system, including the case wherein the fiducials of the medical image comprise the sensors generating the sensor data 302), and the respective data include enough information from which to calculate the respective coordinate system (for example, gait data from inertial sensors may be used to calculate anatomical axes). The registration relationship 310 may be calculated using the respective coordinate frames (of the medical image and sensor data) relative to the same physical anatomical features or axes. In another example, the registration relationship 310 may be calculated by imaging devices or features with a spatial association with the sensors (generating the sensor data), when generating the medical image data 306 (as described above, where the physical sensors (or a subset) may be imaged while coupled to the patient's body in an equivalent manner during which the sensor data was obtained.

### ——Psychometric Features——

As previously mentioned, in accordance with an embodiment, condition data (e.g. first condition data 112A and second condition data 112B) includes psychometric data (e.g. psychometric test results), representing a psychological profile of the patient. An example psychometric test is the SF-36 Health Questionnaire. Other relevant examples may include: the Pain Catastrophization Scale (PCS) and the Hospital Anxiety and Depression Scale (HADS-A/HADS-D). The psychometric data may be provided to the extrapolation module 116, from the prehabilitation module 110. The extrapolation module 116, in accordance with an embodiment, implements a model of the patient that includes psychological features or aspects. The model of the patient may be implemented in similar ways to the previously-described patient models. For example, the psychological model may be implemented as: a trained machine learning system (e.g. based on artificial neural networks); a parameterized model based on physiology, neuroscience, biochemistry or clinical psychology; a statistical or probabilistic model, etc. A patient model may be implemented that includes both the psychological model and the kinetic model (as previously described), as these models may be coupled (for example, the psychological characteristic of post-operative motivation may influence post-operative movement). The extrapolation operation may be performed in similar ways to those previously described, and the result of the extrapolation may include the post-surgical psychological condition of the patient.

In accordance with an embodiment, post-operative psychological condition data includes predicted aspects (e.g. as metrics) such as one or more of: fear, motivation (e.g. motivation for rehabilitation), risk tolerance, anxiety, pain tolerance, expectations (e.g. expectations of outcomes, catastrophization, etc.), and compliance (e.g. compliance with prescribed post-operative protocols).

Post-operative psychological (including mental, emotional, etc.) health affects patient outcomes. For example, there has been demonstrated a link between pain scores and patient anxiety (See Ref. 3 below). The post-surgical psychological condition of the patient may be provided (along with the post-surgical kinetic condition 118) to the surgical planning module 120. The post-surgical psychological condition may influence spatial aspects of surgical planning (as described above the post-surgical psychological condition may influence how the patient moves post-operatively). The post-surgical psychological condition information may be provided to the surgeon via a user interface, such as the warning information 210 of the user interface presented in FIG. 2.

### ——Surgery Scheduling——

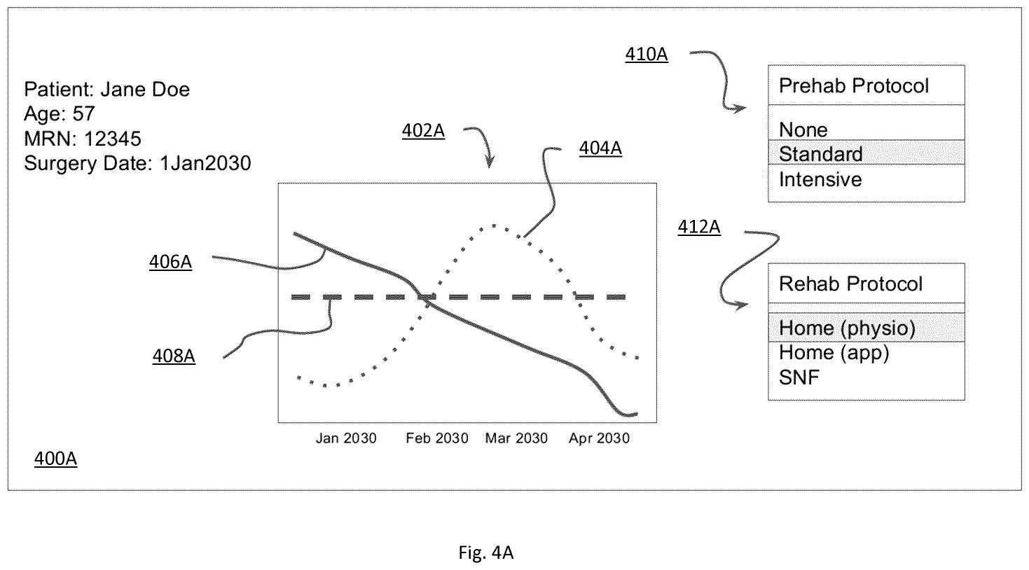

FIGS. 4A and 4B are illustrations of a surgery scheduling user interface 400 and 500, comprising respective instances thereof and in accordance with an embodiment. Further, in accordance with an embodiment the systems described herein are configured to optimize the surgery date. For example, the surgery date may be optimized in any of the following ways (either alone, or in combination with others): minimizing risk of complications; maximizing patient satisfaction; delaying surgery, including delaying indefinitely; minimizing healthcare system resources; triaging or prioritizing a surgeon's wait list; and, optimizing the post-surgical kinetic condition of the patient. As previously described, the surgery date may be a parameter used by the extrapolation module 116 to perform extrapolation operations. The extrapolation module 116 may be optionally configured to perform extrapolations for multiple scenarios based on a varying surgery date. Information to optimize surgery scheduling, based on predicted patient condition data, may be provided to a surgeon via a user interface, such as the user interface shown in FIG. 4A. This user interface shows a graph 402A with a surgical outcome parameter 404A, a non-surgical outcome parameter 406A, and a benchmark parameter 408A, each graphed over time (e.g. the time period associated with candidate surgery dates). The surgical outcome parameter 404A is based at least in part on the post-surgical kinetic condition 118 of the patient, for the given candidate surgery date, and may represent a weighted and/or blended predictive metric for any of: complication risk, patient satisfaction, cost, post-surgical kinetic condition, patient function, etc. The benchmark 408A may represent a threshold wherein outcomes above the threshold are deemed to be worthwhile, whereas outcomes below the threshold are not worthwhile (e.g. the generalized costs outweigh the benefits). Such a threshold may be set by the surgeon based on their practice, or by a payor responsible for paying for the candidate procedure. The non-surgical outcome parameter 406A may be based on the extrapolation module 116 modelling scenarios that involve not operating, for the given date range. The surgical outcome parameter 404A, benchmark 408A and non-surgical outcome parameter 406A may be normalized, such that they can be meaningfully compared on the same graph.

The user interface of FIG. 4A may include data input controls, such as the prehabilitation drop down menu 410A and rehabilitation drop down menu 412A, in which the type of prehabilitation and rehabilitation may be specified by the user (for example, pre-habilitation regimens may be classified as none, standard or intensive, and rehabilitation regimens may be classified as home-based rehabilitation with a physio, home based rehabilitation using a mobile rehabilitation app, or skilled nursing facility). The types of prehabilitation and rehabilitation may be provided to the extrapolation module 116 via the user interface, and invoke the extrapolation module 116 to update its outputs, causing the graph 402A to update. For example, in FIG. 4B (relative to FIG. 4A), the prehabilitation drop down menu 410B indicates a different type of prehabilitation regimen (intensive), and the graph 402B shows an increased date range for viable surgeries (i.e. the surgical outcome parameter 404B being above the benchmark 408B for a larger span of time). Additionally, in this example, the graph 402B indicates that the surgery may be delayed indefinitely, since the non-surgical outcome parameter 406B remains above the benchmark 408B. Other types of data than prehabiliation and rehabilitation types may be fed back to the extrapolation module 116 to cause user interface to update. Rehabilitation drop down menu 412B is the same as menu 412A.

In another example, an alternative surgery outcome parameter is provided; such a parameter may be analogous or similar to the surgical and non-surgical outcome parameters, but represent an alternative treatment. For example, the surgical outcome parameters may represent a total knee arthroplasty surgery, and the alternative surgery outcome parameter may represent a unicondylar knee arthroplasty surgery.

### ——Navigation and Robotics——

In accordance with teachings herein, the prehabilitation-related methods and systems have numerous real word effects. In accordance with an embodiment, the surgical planning module 120 provides registration and target information to a surgical navigation or robotic system. The registration information may include information useful to a surgical navigation or robotic system to register the patient, such that the navigation or robotic system can be used to achieve the desired target, as defined by the target information (e.g. implant position and angle).

A computer device comprises a processor and a storage device coupled thereto, which storage device stores instructions for execution by the processor to configure its operations and that of the computer device so as to perform a method. The method may comprise any of the computer implemented methods as described herein. The computing device typically further comprises an input device and an output device. An input device may comprise any of a keyboard, button, pointing device, microphone, camera, sensor (e.g. GPS or other sensor such as described hereinabove), etc. An output device may comprise a display screen, speaker, light, bell, etc. Some devices provide both input and output functions such as a touch screen device. The computing device further typically comprises a communication subsystem and is configured to communicate such as with coupled input or output devices and/or another computing device via wired or wireless means. The processor may comprise a central processing unit (CPU), graphics processing unit (GPU). The processor may comprise a component of a microcontroller. Storage devices may comprise memory devices including read only memory and random access memory, etc.; hard drives, disc drives, etc. A computer program product comprises a storage device (e.g. a non-transitory device), which stores instructions for execution by a processor of a computing device.

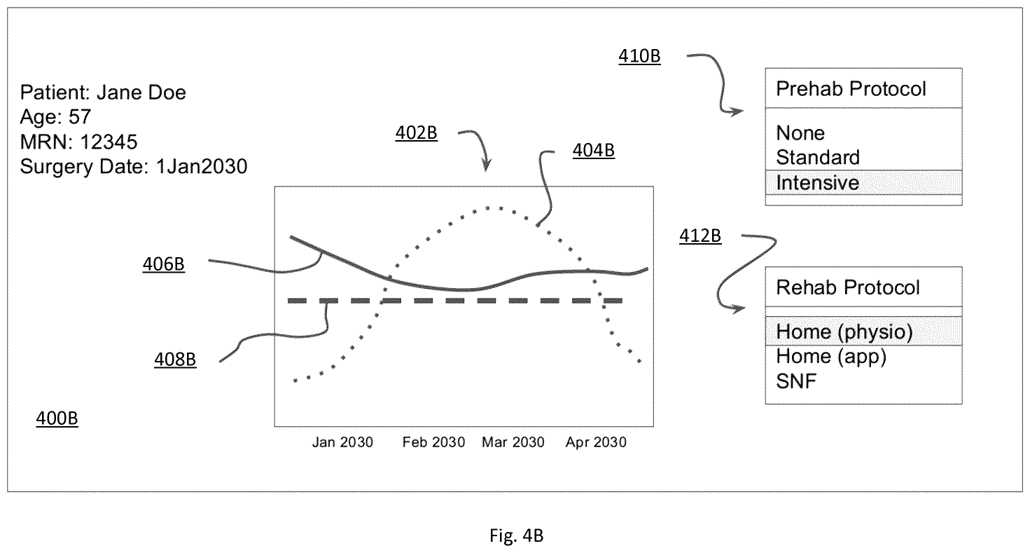

FIG. 5 is a flowchart of operations 500 of a computing device illustrating a computer-implement method. Operations 500 illustrate a computer implemented method for pre-operative planning a total joint arthroplasty for a patient. At 502 operations receive from a prehabilitation system first and second condition data, wherein the first condition data and second condition data comprising kinetic information about how the patient moves from two different time points. In an embodiment, the kinetic information is derived from spatial measurements of the patient's movement; and, the two different time points are separated by sufficient time to allow for detectable physiological changes relating to the patient's kinetic condition. At 504, operations extrapolate a post-surgical kinetic condition of the patient based on the first and second condition data and the planned surgical intervention. At 506, operations calculate a surgical plan optimized for the post-surgical kinetic condition of the patient. At 508, operations provide the surgical plan for display. In an embodiment, (e.g. at 510), the surgical plan is provided for use in a surgical procedure. For example, the plan is provided to a surgical navigation or robotic system for intra-operative execution.

In an embodiment, the condition data further comprises one or more of: demographic information (height, weight, race, gender, BMI, age, etc.); modifiable risk factors (smoking, alcohol, etc.); comorbidities (diabetes); pain scores; prehabilitation compliance metrics; and patient reported outcome measures.

In an embodiment, the kinetic information comprises one or more of: range of motion; gait; posture/stance; and kinematics of Activities of Daily Living (ADL).

In an embodiment, the kinetic information is derived from sensors provided by the prehabilitation system. In an embodiment, the sensors are one or more of: cameras (e.g. phone camera, web camera); motion capture systems (e.g. fiducials attached to patient's body tracked by camera); inertial sensors (accelerometers, gyros, magnetometers); pressure sensors, including pressure mats; and GPS modules.

In an embodiment, the method further comprises calculating and providing additional surgical plans optimized for first and second condition data respectively. In an embodiment, the method further comprises any of: providing the plan and the additional plans for display simultaneously; responsive to user input, updating the display between the plan and additional plans.

In an embodiment, extrapolating is performed in one or more of the following ways: using an extrapolation function whose parameters are determined by a trained model based on a training data set; implementing a parameterized dynamic model of the patient, calculating pre-operative parameters of the first and second condition, calculating a trend in the parameters, calculating updated parameters of the dynamic model based on the surgical intervention and the trend; and, using an optimization method and a generalized patient model.

In an embodiment, calculating the surgical plan is responsive to one or more of: implant impingement; matching pre-operative kinematics; natural movements; and, implant wear.

In an embodiment, the method includes calculating a psychological (psychometric) profile of the patient based on the condition data. Extrapolating is based in part on the psychological profile.

In an embodiment, the extrapolating is further based on future planned surgical interventions.

In an embodiment, the surgical plan includes one or more of the following: implant positions relative to the patient's anatomy; surgical approach; implant sizes; and, implant type (preferably comprising one or more of make and model, high offset, dual mobility, medial pivot, etc.).

In an embodiment, the method further comprises the steps of: extrapolating a post-surgical rehabilitation compliance metric and further calculating the surgical plan based on the post-surgical rehabilitation compliance metric, wherein the post-surgical rehabilitation compliance metric is a predictive parameter for how well the patient will adhere to the prescribed rehabilitation regimen. In an embodiment, the surgical plan includes additional patient preparation for surgery, including one or more of: additional prehabilitation, education, lifestyle and social improvements.

In an embodiment, the pre-operative plan comprises medical imaging of the patient, the medical imaging in a first coordinate system; the kinetic information derived from the sensors providing spatial data in a second coordinate system; and, the method further comprises performing a registration of the first and second coordinate systems, and wherein the pre-operative plan is based in part on the kinetic information expressed relative to the medical image, via the registration. In an embodiment, the method further comprises providing registration information to a robotic or navigation system, wherein the registration information is for a registration process to express the robotic or navigation information relative to the medical imaging.

In an embodiment, the method further comprises the step of calculating: a confidence metric of the extrapolated post-surgical condition, or an uncertainty metric of the extrapolated post-surgical condition, or a range of post-surgical conditions. In an embodiment, the surgical plan provides a range of parameters (e.g. implant types, sizes, positions) based on the range of post-surgical conditions.

In an embodiment, the first and second condition data further comprises one or both of muscle strength information and endurance information In an embodiment, the muscle strength or endurance information comprises a number of repetitions and resistance level of an exercise using an exercise machine or a weight. In an embodiment, the muscle strength or endurance information is measured using a force-measuring device.

In an embodiment, the first and second condition data further comprise cardiovascular health data.

In an embodiment, the first and second condition data further comprise dietary information.

In an embodiment, the method further comprises: receiving a planned surgery date; and extrapolating the post-surgical kinetic condition based on the planned surgery date.

In an embodiment, the surgical plan comprises surgery scheduling information indicative of when the surgery should take place based one or more of: delaying surgery; managing healthcare system resources; prioritizing a surgeon's wait list; optimizing the post-surgical kinetic condition of the patient; and minimizing risk of complications. In an embodiment, the surgical plan includes information regarding an option of delaying surgery indefinitely (e.g. the patient must maintain X weight, Y diet, and tolerate Z pain to delay surgery indefinitely).

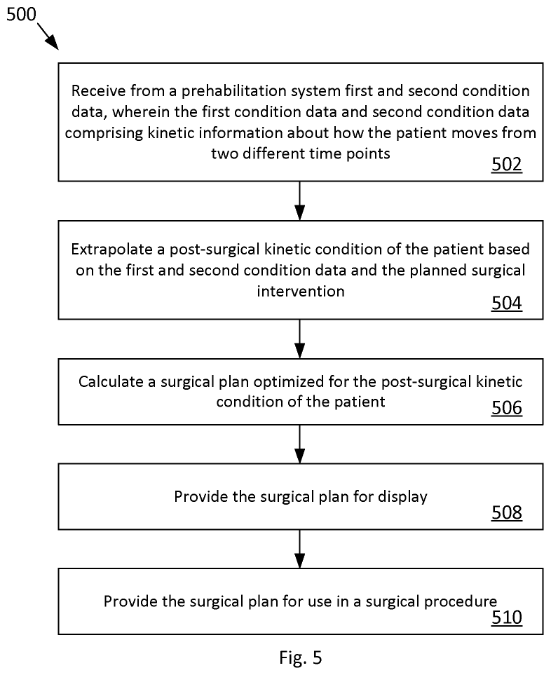

FIG. 6 is a flowchart of operations 600 illustrating a computer implemented method, such as for a pre-habilitation system comprising sensors for generating kinetic information of a patient; and a computer device configured to communicate directly or indirectly with the sensors. The computing device may be configured to operate via instructions stored to a storage device for execution by a processor.

At 602, operations receive sensor data. At 604, operations provide prehabilitation features; and at 606 operations provide first and second condition data to a surgical planning system comprising kinetic information about how the patient moves from two different time points. In an embodiment, the kinetic information is derived from spatial measurements of the patient's movement; and, the two different time points are separated by sufficient time to allow for detectable physiological changes relating to the patient's kinetic condition.

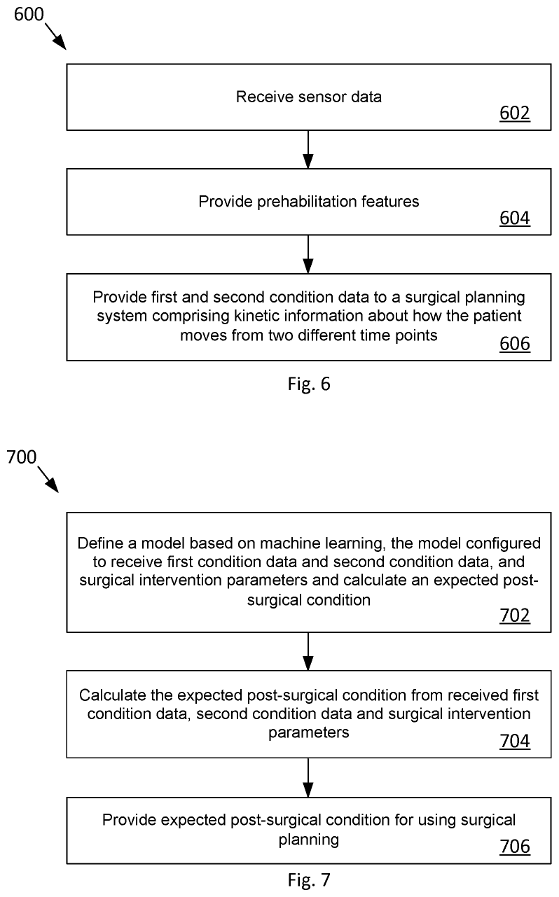

FIG. 7 is a flowchart of operations 700 illustrating a computer implemented method such as for a computing device comprising an extrapolation module. At 702 operations define a model based on machine learning, the model configured to receive first condition data and second condition data, and surgical intervention parameters and calculate an expected post-surgical condition. At 704 operations calculate the expected post-surgical condition from received first condition data, second condition data and surgical intervention parameters. At 706 the expected post-surgical condition is provided for use in surgical planning, for example, by a surgical planning system 114.

In an embodiment, the model comprises a neural network comprising an input layer, one or more hidden layers, and an output layer with weighted connections between nodes in each layer.

In an embodiment, the model comprises a recurrent neural network which receives more than two instances of condition data and processes the instances (e.g. in a sequential nature) to calculate the expected post-surgical condition.

In an embodiment, the model is defined in accordance with machine learning frameworks comprising linear regression, non-linear regression or statistical techniques.

In an embodiment, the expected post-surgical condition comprises information for one or more of: joint range of motion (including the range of motion of a reconstructed joint), standing patient posture, sitting patient posture, parameters of a patient specific dynamic model or activity level (e.g. duration, intensity of post-operative activities).

In an embodiment, the model is configured to receive a surgery date for use in any extrapolation computations.

In an embodiment, the first and second condition data comprise kinetic information about how the patient moves from two different time points. In an embodiment, the kinetic information is derived from spatial measurements of the patient's movement; and the two different time points are separated by sufficient time to allow for detectable physiological changes relating to the patient's kinetic condition.

In an embodiment, the post-surgical kinetic condition is provided to a surgical planning system to define a surgical plan, and, in an embodiment the surgical plan is provided for use in a surgical procedure as described herein.

FIG. 1 shows a computing device configured to perform a method according to any one of the method embodiments herein.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

### REFERENCES (INCORPORATED HEREIN WHERE PERMISSIBLE)

1. E M Keating, C S Ranawat, W Cats-Baril, Assessment of Postoperative Vigor in Patients Undergoing Elective Total Joint Arthroplasty: A Concise Patient- and Caregiver-Based Instrument; Orthopedics 1999 January; 22(1 Suppl):s119-28. URL pubmed.ncbi.nlm.nih.gov/9927112/2.
2. F X McGuigan, W J Hozack, L Moriarty, K Eng, R H Rothman, Predicting quality-of-life outcomes following total joint arthroplasty: Limitations of the SF-36 health status questionnaire; J Arthroplasty 1995 December; 10(6):742-7. doi: 10.1016/s0883-5403(05)80069-5. URL pubmed.ncbi.nlm.nih.gov/8749755/3.
3. T J Wood, P Thornley, D Petruccelli, C Kabali, M Winemaker, J Beer, Preoperative Predictors of Pain Catastrophizing, Anxiety, and Depression in Patients Undergoing Total Joint Arthroplasty; J Arthroplasty 2016 December; 31(12):2750-6. doi 10.1016/j.arth.2016.05.056 URL www.sciencedirect.com/science/article/pii/S0883540316302352

1. A computer implemented method for pre-operative planning a total joint arthroplasty (TJA) for a patient comprising:
receiving from a prehabilitation system first and second condition data, wherein the first condition data and second condition data comprise kinetic information about how the patient moves from two different time points;
wherein the kinetic information is derived from spatial measurements of the patient's movement; and,
wherein the two different time points are separated by sufficient time to allow for detectable physiological changes relating to the patient's kinetic condition;
extrapolating a post-surgical kinetic condition of the patient based on the first and second condition data and the planned surgical intervention;
calculating a surgical plan optimized for the post-surgical kinetic condition of the patient; and
providing the plan for display.

2. The method of claim 1, wherein the condition data further comprises one or more of:
demographic information;
modifiable risk factors;
comorbidities;
pain scores;
prehabilitation compliance metrics;
patient reported outcome measures;
muscle strength;
endurance information;
cardiovascular health; and
dietary information.

3. The method of claim 1, wherein the kinetic information comprises one or more of:
range of motion;
gait;
posture/stance; and
kinematics of Activities of Daily Living (ADL).

4. The method of claim 3, wherein the kinetic information is derived from sensors provided by the prehabilitation system; and wherein the sensors are one or more of:
cameras;
motion capture systems;
inertial sensors;
pressure sensors; and
global positioning system (GPS) sensors.

5. (canceled)

6. The method of claim 1 further comprising:
calculating and providing additional surgical plans optimized for first and second condition data respectively;
providing the plan and the additional plans for display simultaneously; and
responsive to user input, updating the display between the plan and additional plans.

7. (canceled)

8. The method of claim 1, wherein the extrapolating is performed in one or more of the following ways:
using an extrapolation function whose parameters are determined by a trained model based on a training data set;
implementing a parameterized dynamic model of the patient, calculating preoperative parameters of the first and second condition, calculating a trend in the parameters, calculating updated parameters of the dynamic model based on the surgical intervention and the trend; and
using an optimization method and a generalized patient model.

9. The method of claim 1, wherein the calculating the surgical plan comprises one or more of:
implant impingement;
matching pre-operative kinematics;
natural movements; and
implant wear.

10. The method of claim 1, including calculating a psychological profile of the patient based on the condition data, and wherein extrapolating is based in part on the psychological profile.

11. The method of claim 1, wherein the extrapolating is further based on future planned surgical interventions.

12. The method of claim 1 further comprising providing the plan to a surgical navigation or robotic system for intra-operative execution.

13. The method of claim 1, wherein the surgical plan includes one or more of the following:
implant positions relative to the patient's anatomy;
surgical approach;
implant sizes; and
implant type.

14. The method of claim 1 further comprising the steps of:
extrapolating a post-surgical rehabilitation compliance metric and further calculating the surgical plan based on the post-surgical rehabilitation compliance metric, wherein the post-surgical rehabilitation compliance metric is a predictive parameter for how well the patient will adhere to the prescribed rehabilitation regimen.

15. The method of claim 14, wherein the surgical plan includes additional patient preparation for surgery, including one or more of: additional prehabilitation, education, lifestyle and social improvements.

16. The method of claim 4, wherein:
the pre-operative plan comprises medical imaging of the patient, the medical imaging in a first coordinate system;
the kinetic information derived from the sensors providing spatial data in a second coordinate system; and
the method further comprises performing a registration of the first and second coordinate systems, and wherein the pre-operative plan is based in part on the kinetic information expressed relative to the medical image, via the registration.

17. The method of claim 16 further comprising providing registration information to a robotic or navigation system, wherein the registration information is for a registration process to express the robotic or navigation information relative to the medical image.

18. The method of claim 1 further comprising the step of calculating:
a confidence metric of the extrapolated post-surgical condition, or
an uncertainty metric of the extrapolated post-surgical condition, or
a range of post-surgical conditions.

19. (canceled)

20. (canceled)

21. The method of claim 2, wherein muscle strength or endurance information comprises one or both of: a number of repetitions and resistance level of an exercise using an exercise machine or a weight; and measurements using a force-measuring device.

22. (canceled)

23. (canceled)

24. (canceled)

25. The method of claim 1 further comprising:
receiving a planned surgery date; and
extrapolating the post-surgical kinetic condition based on the planned surgery date.

26. The method of claim 25, wherein the surgical plan comprises surgery scheduling information indicative of when the surgery should take place based one or more of:
delaying surgery;
managing healthcare system resources;
prioritizing a surgeon's wait list;
optimizing the post-surgical kinetic condition of the patient;
minimizing risk of complications; and
delaying surgery indefinitely.

27. (canceled)

28. A computing device comprising a processing unit and a storage device coupled thereto, the storage device storing instructions, which when executed by the processing unit, cause the computing device to:
receive from a prehabilitation system first and second condition data, wherein the first condition data and second condition data comprise kinetic information about how the patient moves from two different time points;
wherein the kinetic information is derived from spatial measurements of the patient's movement; and
wherein the two different time points are separated by sufficient time to allow for detectable psychological changes relating to the patient's kinetic condition;
extrapolate a post-surgical kinetic condition of the patient based on the first and second condition data and the planned surgical intervention;
calculate a surgical plan optimized for the post-surgical kinetic condition of the patient; and
provide the plan for display.

29-39. (canceled)
